(19) United States
(12) Patent Application Publication
(10) Pub. No.: US 2022/0323006 A1
(43) Pub. Date: Oct. 13, 2022

(54) COMPLIANT SENSING TETHER FOR IMPLANTABLE BIOSENSOR SYSTEMS

(71) Applicant: W. L. Gore & Associates, Inc., Newark, DE (US)

(72) Inventors: Jérôme S. Conia, Phoenix, AZ (US); Allen B. Mackay, Flagstaff, AZ (US); Benjamin M. Trapp, Phoenix, AZ (US)

(21) Appl. No.: 17/639,159

(22) PCT Filed: Aug. 30, 2019
(86) PCT No.: PCT/US2019/049073

§ 371 Date: Feb. 28, 2022

(51) Int. Cl. A61B 5/00; A61B 5/1459

(52) U.S. Cl. CPC A61B 5/686; A61B 5/1459; A61B 5/4839; A61B 5/14532

(57) ABSTRACT

Embodiments of the present disclosure relate to implantable biosensors configured to be implanted into tissue of a subject at an implantation site. In an exemplary embodiment, the implantable biosensor comprising: an electronic module and a compliant sensing tether extending from the electronic module. The compliant sensing tether comprising a proximal portion coupled to the electronic module, a distal portion spaced apart from the electronics module, and an intermediate portion joining the proximal portion to the distal portion. The proximal portion has a first flexibility and the distal portion having a second flexibility. The second flexibility of the distal portion being greater than the first flexibility of the proximal portion. The distal portion comprises a sensor configured to sense a signal corresponding to an analyte of the subject, wherein the signal corresponding to the analyte is transferred to the electronics module via the compliant sensing tether.

### CROSS-REFERENCE TO RELATED APPLICATION

This application is a national phase application of PCT Application No. PCT/US2019/049073, internationally filed on Aug. 30, 2019, which is herein incorporated by reference in its entirety for all purposes.

### FIELD

The present disclosure relates generally to implantable biosensors. More specifically, the present disclosure relates to implantable biosensors including a compliant sensing tether.

### BACKGROUND

An implantable biosensor is used to sense analytes of a subject. Analytes can provide information about a subject's health, they can be used to assess various dynamic physiological properties driven by either endogenous or environment sources, and they can be used to monitor disease progression (e.g., cardiovascular, excretory, digestive, endocrine, etc.). One noteworthy example consists of subjects diagnosed with insulin dependent diabetes. In these cases, monitoring blood or interstitial glucose levels is important for a subject on insulin therapy to ensure adequate doses of insulin are being administered. Reliable glucose monitoring with minimal lag time and sufficient accuracy is particularly important when administering insulin.

To sense the analytes, the implantable biosensor is arranged in the tissue of a subject, e.g., subcutaneous tissue of a subject. Implantable biosensors, however, elicit a foreign body response by the tissue. The amount of foreign body response elicited by the tissue can vary depending on the characteristics of the implantable biosensors.

### SUMMARY

The present disclosure relates an implantable biosensor including a compliant sensing tether to reduce the amount of foreign body response elicited by tissue. Exemplary embodiments include but are not limited to the following examples.

In an exemplary embodiment, an implantable biosensor configured to be implanted into tissue of a subject at an implantation site, comprises: an electronic module; a compliant sensing tether extending from the electronic module, the compliant sensing tether comprising a proximal portion coupled to the electronic module, a distal portion spaced apart from the electronics module, and an intermediate portion joining the proximal portion to the distal portion; the proximal portion having a first flexibility and the distal portion having a second flexibility, the second flexibility of the distal portion being greater than the first flexibility of the proximal portion; and the distal portion comprising a sensor configured to sense a signal corresponding to an analyte of the subject, wherein the signal corresponding to the analyte is transferred to the electronics module via the compliant sensing tether.

In another exemplary embodiment, a method for monitoring an analyte of a subject, comprises: inserting an implantable biosensor into an implantation site of the subject, the implantable biosensor comprising: an electronic module; a compliant sensing tether extending from the electronic module, the compliant sensing tether comprising a proximal portion coupled to the electronic module, a distal portion spaced apart from the electronics module, and an intermediate portion joining the proximal portion to the distal portion; the proximal portion having a first flexibility and the distal portion having a second flexibility, the second flexibility of the distal portion being greater than the first flexibility of the proximal portion; the distal portion comprising a sensor configured to sense a signal corresponding to an analyte of the subject; sensing, by the sensor, the signal corresponding to the analyte of the subject; and transferring, via the compliant sensing tether, the signal to the electronics module.

In example thereof, further comprising transmitting, by the electronic module, the signal to an external device.

In another example thereof, further comprising analyzing, by the electronic module, the signal to determine an amount of analyte in the subject.

In yet another example thereof, the second flexibility of the distal portion being substantially equal to or less than a predetermined flexibility of the tissue at the implantation site.

In a further example thereof, the compliant sensing tether having a stiffness gradient that decreases nonlinearly from the proximal portion to the distal portion.

In another example thereof, the compliant sensing tether having a stiffness gradient that decreases linearly from the proximal portion to the distal portion.

In yet another example thereof, the compliant sensing tether having a flexibility that increases in response to fluid absorption by the compliant sensing tether.

In another example thereof, the distal portion comprising a plurality of sensors.

In yet another example thereof, the distal portion being formed from ePTFE.

In another example thereof, the electronics module comprising an antenna, a battery, and a circuit board.

In yet another example thereof, the compliant sensing tether having a stepped compliance.

In even another example thereof, the compliant sensing tether having one or more of the following characteristics: a tensile strength less than 50 kPa, a toughness modulus less than 50 kPa, and a flexibility less than 50 kPa.

In yet even another example thereof, the distal portion having a compressive modulus less than 35 kPa.

In another example thereof, the compliant sensing tether configured to dispose a hydrogel proximate to the distal portion of the compliant sensing tether.

In yet another example thereof, the electronic module configured to sense a fluorescence of the hydrogel.

In even another example thereof, the compliant sensing tether being separate from the electronic module and wherein the compliant sensing tether transmits sensor signals to the electronic module.

In yet another example thereof, the compliant sensing tether being separable from the electronic module.

In even another example thereof, the compliant sensing tether being coated in a hydrogel.

In yet another example thereof, the implantable biosensor being incorporated into a therapeutic drug infusion pump.

In another exemplary embodiment, a method of treatment using an implantable biosensor, comprises: receiving sensed signals from the implantable biosensor implanted in a subject; the implantable biosensor comprising:

an electronic module; a compliant sensing tether extending from the electronic module, the compliant sensing tether comprising a proximal portion coupled to the electronic module, a distal portion spaced apart from the electronics module, and an intermediate portion joining the proximal portion to the distal portion; the proximal portion having a first flexibility and the distal portion having a second flexibility, the second flexibility of the distal portion being greater than the first flexibility of the proximal portion; and the distal portion comprising a sensor configured to sense a signal corresponding to an analyte of the subject, wherein the signal corresponding to the analyte is transferred to the electronics module via the compliant sensing tether; processing the received signals to determine concentration of the analyte; and sending a signal to a therapy device to provide treatment based on the determined concentration.

In an example thereof, further comprising implanting the implantable biosensor in the subject.

The foregoing Examples are just that and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a schematic illustration of a system including an implantable biosensor having a compliant sensing tether, in accordance with embodiments of the present disclosure.

FIG. 2 is an illustration of an implantable biosensor having a compliant sensing tether, in accordance with embodiments of the present disclosure.

FIG. 3 is an illustration of an implantable biosensor having a compliant sensing tether implanted within a portion of a subject, in accordance with embodiments of the present disclosure.

FIG. 4 is a perspective view of an exemplary implantable biosensor having a compliant sensing tether, in accordance with embodiments of the present disclosure.

FIG. 5 is a perspective view of another exemplary implantable biosensor having multiple compliant sensing tethers, in accordance with embodiments of the present disclosure.

FIG. 6 is a perspective view of an exemplary implantable biosensor having a detachable compliant sensing tether, in accordance with embodiments of the present disclosure.

FIG. 7 is a perspective view of an exemplary implantable biosensor having a wireless compliant sensing tether, in accordance with embodiments of the present disclosure.

FIG. 8 is a perspective view of an exemplary compliant sensing tether, in accordance with embodiments of the present disclosure.

FIG. 9 is a side view of an exemplary compliant sensing tether and exemplary flexibility gradients associated therewith, in accordance with embodiments of the present disclosure.

FIG. 10 are cross-sectional views of exemplary shapes of compliant sensing tethers, in accordance with embodiments of the present disclosure.

FIG. 11 is a perspective view of another exemplary compliant sensing tether, in accordance with embodiments of the present disclosure.

FIG. 12 is a perspective view of even another exemplary compliant sensing tether, in accordance with embodiments of the present disclosure.

FIG. 13 is a perspective view of even another exemplary compliant sensing tether, in accordance with embodiments of the present disclosure.

FIG. 14 is a perspective view of even another exemplary compliant sensing tether, in accordance with embodiments of the present disclosure.

FIG. 15 is a perspective view of even another exemplary compliant sensing tether, in accordance with embodiments of the present disclosure.

FIG. 16 is a perspective view of even another exemplary compliant sensing tether, in accordance with embodiments of the present disclosure.

FIG. 17 is a perspective view of even another exemplary compliant sensing tether, in accordance with embodiments of the present disclosure.

FIG. 18 is a perspective view of even another exemplary compliant sensing tether, in accordance with embodiments of the present disclosure.

FIG. 19 is a perspective view of even another exemplary compliant sensing tether, in accordance with embodiments of the present disclosure.

FIG. 20 is a perspective view of an exemplary sensing region of a compliant sensing tether, in accordance with embodiments of the present disclosure.

FIG. 21 is a perspective view of another exemplary sensing region of a compliant sensing tether, in accordance with embodiments of the present disclosure.

FIG. 22 is a perspective view of even another exemplary sensing region of a compliant sensing tether, in accordance with embodiments of the present disclosure.

FIG. 23 is a perspective view of even another exemplary sensing region of a compliant sensing tether, in accordance with embodiments of the present disclosure.

FIG. 24 is a perspective view of even another exemplary sensing region of a compliant sensing tether, in accordance with embodiments of the present disclosure.

As the terms are used herein with respect to ranges of measurements “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like. Additionally, with respect terminology of inexactitude, the terms “about” and “approximately” may be used, interchangeably, to refer to a measurement that includes the stated measurement and that also includes any measurements that are reasonably close to the stated measurement. In the event it is determined that individuals having ordinary skill in the relevant arts would not readily ascertain values for such reasonably small differences, the terms “about” and “approximately” can be understood to mean plus or minus 10% of the stated value.

This disclosure is not meant to be read in a restrictive manner. For example, the terminology used in the application should be read broadly in the context of the meaning those in the field would attribute such terminology.

Certain terminology is used herein for convenience only. For example, words such as “top”, “bottom”, “upper,” “lower,” “left,” “right,” “horizontal,” “vertical,” “upward,” and “downward” merely describe the configuration shown in the figures or the orientation of a part in the installed position. Indeed, the referenced components may be oriented in any direction. Similarly, throughout this disclosure, where a process or method is shown or described, the method may be performed in any order or simultaneously, unless it is clear from the context that the method depends on certain actions being performed first.

### DETAILED DESCRIPTION

Persons skilled in the art will readily appreciate that various aspects of the present disclosure can be realized by any number of methods and apparatus configured to perform the intended functions. It should also be noted that the accompanying drawing figures referred to herein are not necessarily drawn to scale but may be exaggerated to illustrate various aspects of the present disclosure, and in that regard, the drawing figures should not be construed as limiting.

As stated above, the amount of foreign body response eliciting by the tissue can vary depending on the characteristics of the implantable sensors. For example, local inflammation, the development of scar tissue, the presence of seroma, and/or the development of a foreign body capsule in response to sensor implantation constitute persistent obstacles to the advancement of long-term in vivo bio-marker monitoring solutions. The body's reaction to the presence of a foreign entity (e.g., an implanted sensor) is a natural phenomenon driven by the immune system over time. This reaction leads to the progressive encapsulation of the implantable sensor with reduced transport of analytes towards the sensor surface hindering the functionality of the sensor and, therefore, rendering the system ineffective. Some embodiments have attempted to solve some of these issues by developing bio-sensor systems without an onboard power source and electronics. However, these solutions present a deficit in autonomy and functionality because these systems require components to remain external to the body that must be attached to the skin with an adhesive patch. Embodiments disclosed herein alleviate these issues by including an implantable biosensor having a compliant sensing tether that reduces the foreign body response in comparison to conventional embodiments.

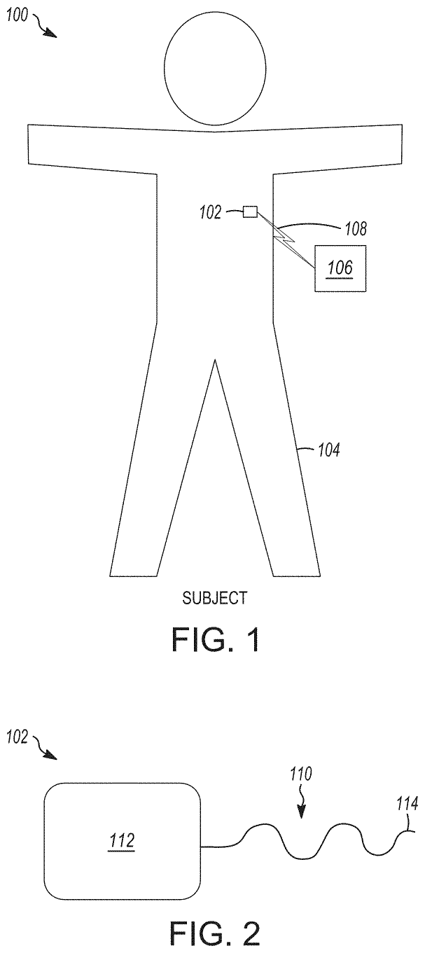

FIG. 1 is a schematic illustration of a system 100 including an implantable biosensor 102 having a compliant sensing tether, in accordance with embodiments of the present disclosure. As shown in FIG. 1, the implantable biosensor 102 is configured to be arranged within the body of a subject 104 to sense one or more analytes of the subject 104. The one or more analytes can then be analyzed to, for example, determine various dynamic physiological properties of the subject 104 driven by either endogenous or environment sources, monitor disease progression of the subject 104, detect ailments or infections of the subject 104, detect transient physiological parameters or trends of the subject 104, monitor a therapeutic or training regimen of the subject 104, and/or the like. Additionally, or alternatively, the implantable biosensor 102 may be used to monitor fluid dynamics, hydration, temperature variations, metabolite concentrations, muscle kinetics, neuronal signaling, orthopedic parameters, and/or used in association with the delivery of targeted stimulations, either chemical, hormonal, optical or electrical, as they may be needed in the treatment of various disorders (e.g. drug-eluting contraptions or scaffolds, artificial body parts or organs, etc.).

One exemplary application for the implantable biosensor 102 may be for the detection of pressure such as vascular pressure for the screening of hypertension, or for assessing either occupational, cardiovascular, or surgical risks. Other exemplary applications may include the detection of the rate of mechanical tissue deformations or in the detection of sounds or ultrasound, such as sounds originating from circulating fluids, turbulences and/or oscillations. Further exemplary applications may be the field of acoustic reflectometry when wave monitoring is used for clinical determinations. Additionally, or alternatively, the implantable biosensor 102 may be used in the examination of nerve conduction and electrical impulse frequency, which may have impact in the diagnosis and treatment of various neurological disorders, or which may be related to muscular activities. The implantable biosensor 102 may also be used to measure core or superficial body temperature, which may have predictive value, with regards to the symptoms associated with multiple sclerosis or epilepsy. Further use of the implantable biosensor 102 may be monitoring of body hydration, which is of particular importance for seniors or otherwise impaired patient populations. Other exemplary applications of the implantable biosensor 102 may include the detection of bio-markers (e.g. analytes) representative of congestive heart failure as a means to anticipate decompensation course and prevent or limit occurrence of patient hospitalization. Even other exemplary applications of the implantable biosensor 102 may include the detection of various molecular clues and organic compounds that inform about physiological conditions, such as lipids, proteins, or carbohydrates, for example glucose as it relates to diabetes.

In exemplary embodiments, the implantable sensor 102 senses analytes indicative of body characteristics on a continuous, intermittent or near continuous basis. The implantable biosensor 102 may be an electrochemical and/or photonic sensor that uses enzymatic and/or optical properties to sense analyte concentrations in the interstitial fluid. For example, glucose levels may be determined by the implantable biosensor 102 using electrodes and glucose oxidase. Other exemplary analytes include, but are not limited to, glucose, potassium, inorganic phosphorous, magnesium, lactate dehydrogenase (LD), lactate, oxygen, insulin, C-peptide, parathyroid hormone (PTH), osteocalcin, C-telopeptide, brain natriuretic peptide (BNP), adrenocorticotropic hormone (ACTH), other types of hormones, pharmacologic agents, bio-pharmaceuticals, proteins and peptides, biomarkers, antibodies, therapeutic agents, electrolytes, vitamins, pathogenic components, antigens, molecular markers associated with different disease conditions in stages, viral loads, and/or the like.

In embodiments, the implantable biosensor 102 is configured to be communicatively coupled to an external device (ED) 106 via a communication link 108. The ED 106 may be configured to receive, store, and/or process signals (e.g., analyte concentrations) sensed by the implantable biosensor 102. Additionally, or alternatively, the ED 106 may administer therapy via, for example, chemical, hormonal, or electrical stimulations (e.g. a medicine or insulin pump), based on the signals sensed by the implantable biosensor 102. In at least some embodiments, the ED 106 may also perform a power management function for the implantable biosensor 102. For example, the ED 106 may wake the implantable biosensor 102, sleep the implantable biosensor 102, and/or direct the implantable biosensor 102 to sense, store, process, and/or transmit signals. Embodiments of the ED 106 may be any type of device having computing capabilities such as, for example, a smartphone, a tablet, a notebook, or other portable or non-portable computing device.

The communication link 108 may be, or include, a wired link (e.g., a link accomplished via a physical connection) or a non-wired link such as, for example, a short-range radio link, such as Bluetooth, IEEE 802.11, near-field communication (NFC), WiFi, a proprietary wireless protocol, and/or the like. The term “communication link” may refer to an ability to communicate some type of information in at least one direction between at least two devices and should not be understood to be limited to a direct, persistent, or otherwise limited communication channel. That is, according to embodiments, the communication link 108 may be a persistent communication link, an intermittent communication link, an ad-hoc communication link, and/or the like. The communication link 108 may refer to direct communications between the implantable biosensor 102 and the ED 106, and/or indirect communications that travel between the implantable biosensor 102 and the ED 106 via at least one other device (e.g., a repeater, router, hub, and/or the like). The communication link 108 may facilitate uni-directional and/or bi-directional communication between the implantable biosensor 102 and the ED 106. Data and/or control signals may be transmitted between the implantable biosensor 102 and the ED 106 to coordinate the functions of the implantable biosensor 102 and/or the ED 106. In embodiments, subject data may be downloaded from one or more of the implantable biosensor 102 and the ED 106 periodically or on command. The clinician and/or the subject 104 may communicate with the implantable biosensor 102 and/or the ED 106, for example, to initiate, terminate and/or modify sensing, storing, processing, and/or transmitting signals.

FIG. 2 is an illustration of an implantable biosensor 102 having a compliant sensing tether 110 and an electronic module 112 communicatively coupled to the compliant sensing tether 110, in accordance with embodiments of the present disclosure. The compliant sensing tether 110 may exhibit uniform or variable mechanical properties along its length, which may match the mechanical properties of the tissue in which the implantable biosensor 102 is implanted.

Arranged on a distal end of the compliant sensing tether 110 is at least one sensing region 114 configured to sense one or more analytes. While one sensing region 114 is depicted, the compliant sensing tether 110 may include more sensing regions 114. The sensing region 114 may be coupled to and/or integrated in a highly compliant section of the compliant sensing tether 110, e.g., at the distal portion of the compliant sensing tether 110. In at least some embodiments, the analytes sensed by the sensing region 114 may be transferred to the electronics module 112 via the compliant sensing tether 110. Because an adverse foreign body response by the tissue should be avoided around the sensing region 114 itself, the sensing region 114 may be the smallest at the surface of a highly compliant section of the compliant sensing tether 110, thereby allowing minimally invasive placement within the target interstitial compartment in order to reduce, minimize, or evade adverse body responses.

To reduce, minimize, or evade adverse body responses, the stress-strain behaviors (e.g., flexibility) of the sensing region 114 may be similar to or match the host tissues in which the implantable biosensor 102 is implanted or the host tissues in which the sensing region 114 is implanted. For instance, the mechanical properties of the sensing region 114 may be inferior to, equivalent, or similar to that of the surrounding live tissues. The physical properties of the sensing region 114 may be designed for minimizing tissue injury and foreign body response, as well as for minimal adverse impact on capillary density, all of which could degrade the implantable biosensor's 102 ability to function as a long-term bio-marker monitoring solution.

To achieve desired physical properties, the compliant sensing tether 110 and the sensing region 114 may be constructed with biocompatible and microporous materials, thin composite films or engineered microstructures with controlled porosity such as polytetrafluoroethylene or expanded polytetrafluoroethylene (ePTFE), flexible elastomeric polymers, carbon loaded film, solid or stranded wires, printed circuit, flexible circuit and micro-flat pipes, hydrophobic or hydrophilic coating for either enhanced insulation or contact.

In at least some embodiments, the compliance of the compliant sensing tether 110 may increase from the proximal region near the electronic module 112 to the distal region near the sensing region 114 as explained in more detail below. Additionally, the compliant sensing tether 110 may have an elongated shape that perm its the sensing region 114 to be physically spaced apart from the electronic module 112 so the sensing region 114 and electronic module 112 can be arranged in two distinct locations, either within the same tissue layer or across different tissue beds or body compartments. The electronic module 112 may include a power storage unit and/or an antenna, which may be used to communicate wireless with the ED 106.

It is expected that soft tissues will be exposed to mechanical stress following implantation of the implantable biosensor 102. Tissue deformation and irritation will be proportional to the implantable biosensor 102 size and mismatch between tissue and biosensor physical properties. The implantable biosensor 102 has been chosen to allow the sensing region 114 itself to reduce the invasiveness, with the compliant sensing tether 110 and the sensing region 114 moved away from the electronic module 112 where the foreign body response is most pronounced. Furthermore, the physical properties of the implantable biosensor 102 are intended to reduce the occurrence and impact of foreign body response around the sensing region 114.

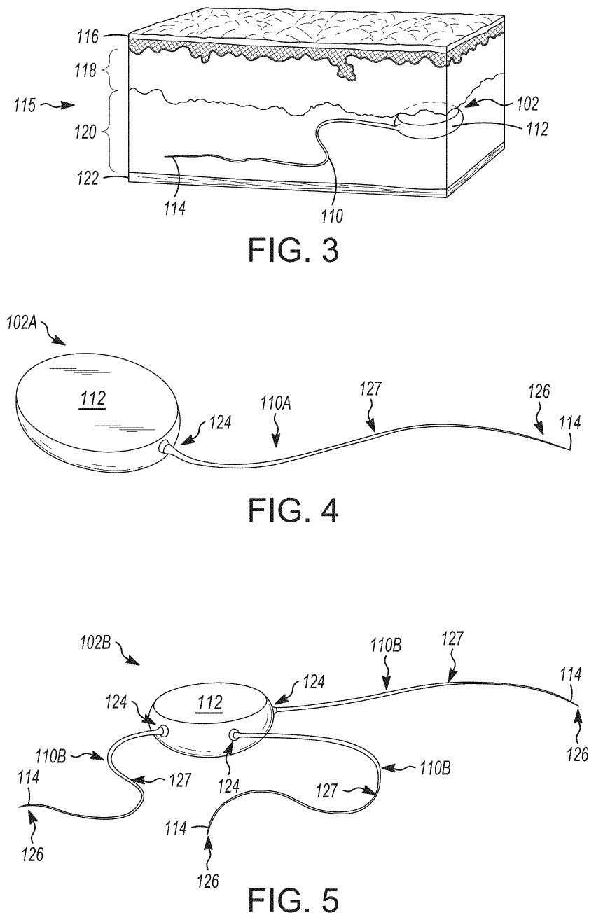

FIG. 3 is an illustration of an implantable biosensor 102 having a compliant sensing tether 110 implanted within tissue 115 of a subject 104, in accordance with embodiments of the present disclosure. Surgical and/or interventional tools such as trocars, tunnelers, forceps, needles, tubes, sheaths, catheters, or other insertion guides may be used to implant the implantable biosensor 102 into the subject 104. In at least some embodiments, the electronic module 112 may be implanted beneath the epidermis 116 close to dense irregular connective tissues of the dermis 118 and/or in a superficial sub-cutaneous layer 120 position above the muscle layer 122. Placement of the electronic module 112 in a rather superficial position helps efficient data transfer to the ED 106 and/or helps facilitate induction charging of the implantable biosensor 102 due to the minimal amount of biomaterial on top of it. The electronic module 112 placement also corresponds to less invasive procedures for both implantable biosensor 102 insertion and removal. In at least some embodiments, foreign body encapsulation may help hold the electronic module 112 enclosure in place.

The length of the compliant sensing tether 110 permits placement of the sensing region 114 in a different tissue plane, tissue bed or body compartment than the electronic module 112, for example where the capillary density is greater. The susceptibility of at least a length portion of the compliant sensing tether 110 and of the sensing region 114 to undergo plastic deformation under load at the site of implantation allows the sensing region 114 to mold itself onto the host tissue 115. The physical properties of the compliant sensing tether 110 and sensing region 114 enable a quiet biological response and facilitate the founding of a stable bio-interface with the host tissues 115.

Additionally, due to the characteristics of the implantable biosensor 102, it becomes possible to implant the compliant sensing tether 110 within the intra-peritoneal space. The purpose of this configuration is to enable increased accuracy of detection of analytes with negligible lag.

More specifically, the peritoneum is a thin membrane consisting of two layers of mesothelial cells, lining the abdominal wall and the abdominal viscera, wrapping around the internal organs. The peritoneal cavity is the potential space defined by the gap separating these two layers. The parietal and visceral layers of the peritoneum constantly produce and resorb a thin film of fluid. The peritoneal fluid remains in equilibrium with the blood plasma and with the interstitial fluid from adjacent tissues; it contains water, electrolytes and various metabolites as well as offers an accurate representation of the circulating blood constituents. Because of the large combined surface area of the parietal and visceral layers, peritoneal fluid regeneration occurs rather quickly which makes it an advantageous route for the administration of certain pharmacotherapies and for the continuous monitoring of analytes.

Due to the ability to implant the compliant sensing tether 110 in an intra-peritoneal region, signal accuracy is increased because the dynamics of analytes (e.g., bio-marker populations and molecular species) transported within the intra-peritoneal fluid closely mimic physiological parameters. Signal quickness is also increased due to the rapid fluid regeneration within the intra-peritoneal cavity as compared to the sub-cutaneous interstitial fluid. Physiological and physical time delays may be decreased, moderating the overall monitoring lag time compared to the next best alternatives.

For implantation of the implantable biosensor 102 within the intra-peritoneal space, the electronic module 112 may be arranged in the abdominal area. The compliant sensing tether 110 may be passed through the parietal layer of the peritoneum and the sensing region 114 may be immerged in the intra-peritoneum fluid, where analytes of interest may be present. Intra-peritoneal access requires careful aseptic conditions to circumvent risk of peritonitis. The low profile of the compliant sensing tether 110 and sensing region 114 contributes to reducing the invasiveness of the placement of the implantable biosensor 102, thereby reducing the likelihood of damage to organs. This placement may facilitate frictionless movement between the parietal and visceral layers during contraction and relaxation of the digestive tract muscles.

The high compliance of the compliant sensing tether 110 and particularly of the sensing region 114 constitutes a contributor to enabling the long-term effectiveness of the implantable biosensor 102. The low stiffness of the compliant sensing tether 110 may be guided by the bulk material properties of the target host tissues 115. In at least some embodiments, the compliant sensing tether 110 should remain compliant under minor stress and its functionality should not be impaired by deformation or stretching. The compliant sensing tether 110 may have high resiliency, storing energy under elastic deformation, or exhibit plasticity, dissipating energy and deforming permanently when subjected to a load that exceeds its elastic limit. Other possible contributors to the long-term effectiveness of the implantable biosensor 102 may reside in the material properties of the compliant sensing tether 110 components. The moduli of each material component within the compliant sensing tether 110 may be engineered to eliminate or reduce mechanical mismatch with the host tissue 115 at the sensing region 114. Preferably, the compliance of the compliant sensing tether 110 and sensing region 114 should either match or exceed the compliance of the host tissue 115. Material porosity in the appropriate scale may enable molecular transport while tailoring cell ingrowth for facilitated extraction. Hydrophilic treatment may be incorporated into the compliant sensing tether 110 (e.g., the sensing region 114) to facilitate recruitment of water molecules for implantable biosensor 102 hydration.

The host tissue 115 in which the implantable biosensor 102 is implanted is viscoelastic and may consist of non-linear and anisotropic materials. These intricate physical properties are influenced by complex structures including proteoglycan matrices, elastic and collagen fibers. An implantable device (e.g., the implantable biosensor 102) may induce time-dependent nonlinear stresses and strains in tissues 115. A typical force-deformation curve for a biological specimen is nonlinear and exhibits different responses based on the magnitude of deformation. When a biological material is exposed to a large strain, the stress-strain curve passes the yield point (or elastic limit of the material), which corresponds to the point at which some fraction begins to deform plastically (permanently), eventually reaching irreversible fiber failure or fracture if the stretch is substantial. The scenario of tissue 115 plastic behavior must be controlled to secure successful and durable bio-sensing. Hence, the compliant sensing tether 110 and sensing region 114 of the implantable biosensor 102 may be designed to result in minimal host tissue stretch. Below the yield point, the slope of the stress-strain curve represents the elastic modulus of the tissue 115. Under those conditions, the tissue 115 is elastic; it deforms but retains the ability to regain its original shape at removal of the stress. Biological materials having a high elastic modulus require more force to stretch.

In at least some embodiments, the sensing region 114 may be highly compliant. Its physical properties may be designed to be in harmony with the nonlinear or toe region of a typical force-deformation curve for a biological host tissue 115. For this reason, at least a length portion of the sensing region 114 exhibits high flexibility and/or low stiffness.

The graph illustrated below is the stress-strain curve of human skin. A similar graph is provided by Yu J, Dinsmore R, Masoumy M, Seqoia J, Baban, B. in “An Integrative Approach to Chronic Wounds in Patients with Diabetes: PPPM in Action. New Strategies to Advance Pre/Diabetes Care: Integrative Approach” by PPPM (pp. 283-321), January 2013, the entire contents of which are incorporated herein for all purposes. This curve shows the viscoelastic behavior with initial “toe-in” (viscous) phase followed by the linear (elastic phase) leading up to permanent (plastic) deformation and eventually to breaking (failure).

In at least some embodiments, the sensing region 114 and host tissue 115 constitute a coupled system with persistent contact that may remain compliant over an extended period of time (multiple months or years) for a variety of tissue conditions (young vs. old; healthy vs. diseased; level of hydration; gender) or environmental conditions (gravity; level of physical activity) that may mimic the stress-strain curve of human skin illustrated in the graph above. In at least some embodiments, the design, and the shape of the sensing region 114 may allow fora minimally disruptive distribution of the sensing region 114 volume in all directions within the host tissues 115. The compliant sensing tether 110 and the sensing region 114 may be intended to elicit minimum strain anywhere below the yield point of the host tissue 115.

Upon surgical placement of the implantable biosensor 102, for example using a trocar or equivalent implantation tools, the host tissue 115 will experience instant deformation. Subsequently, the implantable biosensor 102 may exert a time-dependent load on the tissue 115. A balanced force may be exerted by the tissue 115 on the implantable biosensor 102. In addition, stress resulting from the gravitational forces on the implantable biosensor 102 as well as the forces associated with accelerations accompanying a patient's daily kinetic activities will be proportional to the implantable biosensor 102 mass. Because the implantable biosensor 102 is intended to be durable over periods of time ranging from several months to years, it is important to reduce long-term stress for the purpose of minimizing time-dependent tissue 115 strain. The amount of tissue 115 deformation should be kept minimum. This is made possible by the miniaturized sensing region 114 size and weight. In exemplary embodiments, the amount of load exerted by the sensing region 114 may be intended to be negligible compared to the material properties (viscoelasticity, stiffness) of the host tissue 115.

FIG. 4 is a perspective view of an exemplary implantable biosensor 102A having a compliant sensing tether 110A, in accordance with embodiments of the present disclosure. In the illustrated embodiment, the implantable biosensor 102A comprises a single compliant sensing tether 110A having a proximal portion 124, a distal portion 126, and an intermediate portion 127 connecting the proximal portion 124 to the distal portion 126. Further, a proximal portion 124 of the compliant sensing tether 110A is also hard-wired to the electronic module 112. Additionally, the size of the compliant sensing tether 110A has a variable size such that the cross-sectional area decreases from the proximal portion 124 to the distal portion 126 of the compliant sensing tether 110A. As a result, the flexibility of the compliant sensing tether 110A increases from the proximal portion 124 to the distal portion 126. The distal portion 126 corresponds to the compliant area where the at least one sensing region 114 is located. As stated above, the at least one sensing region 114 detects one or more analytes. In at least some embodiments, the electronic module 112 communicates wirelessly with a receiver external to the body (e.g., the ED 106). This embodiment allows for stability of the implantable biosensor 102A and allows for simultaneous implantation of all components of the implantable biosensor 102A in a single surgical procedure.

FIG. 5 is a perspective view of another exemplary implantable biosensor 102B having multiple compliant sensing tethers 1106, in accordance with embodiments of the present disclosure. Specifically, the illustrated embodiment includes three compliant sensing tethers 1106 with at least three respective sensing regions 114. Similar to the embodiment depicted in FIG. 4, the flexibility of the compliant sensing tethers 1106 may increase from respective proximal portions 124 of the compliant sensing tethers 1106 to distal portions 126 of the compliant sensing tethers 1106. Moreover, the proximal portions 124 may be hard-wired to the electronic module 112 and the distal portions 126 correspond to the compliant areas where the sensing regions 114 are located. In at least some embodiments, the electronic module 112 may communicate wirelessly with a receiver external to the body (e.g., the ED 106).

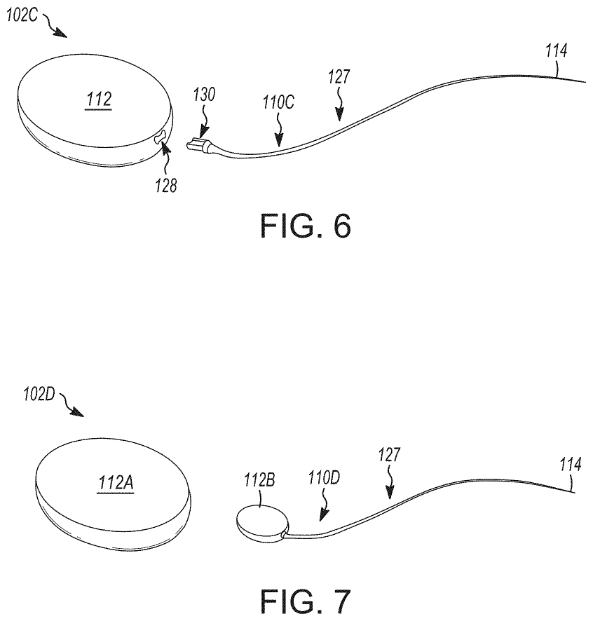

FIG. 6 is a perspective view of an exemplary implantable biosensor 102C having a detachable compliant sensing tether 110C, in accordance with embodiments of the present disclosure. The illustrated implantable biosensor 102C comprises an electronic module 112 and a single compliant sensing tether 110C with at least one sensing region 114. In addition, the electronic module comprises a connection port 128 that couples with a connector 130 of the compliant sensing tether 110. This embodiment allows separate packaging, storage, implantation, and/or removal of the electronic module 112 and the compliant sensing tether 110.

FIG. 7 is a perspective view of an exemplary implantable biosensor 102D having a wireless compliant sensing tether 110D, in accordance with embodiments of the present disclosure. The illustrated embodiment includes two electronic modules 112A, 112B. The first electronic module 112A may be larger in size and may contain a power source (battery or rechargeable battery), antenna, and electronic board, but is not physically connected to the sensing tether 110D. The first electronic module 112A may communicate wirelessly with the second electronic module 1126 by means of RF energy. The second electronic module 112B, which may be smaller in size than the first electronic module 112A, may act as a relay. In at least some embodiments, the second electronic module 1126 may not have an on-board power source. The second electronic module 112B may be hard-wired with the compliant sensing tether 110D and sensing region 114. The absence of any physical contact between the two electronic modules 112A, 112B may reduce the likelihood of mechanical interference therebetween. Furthermore, this embodiment may allow for sequential implantation and removal in the course of separate surgical procedures.

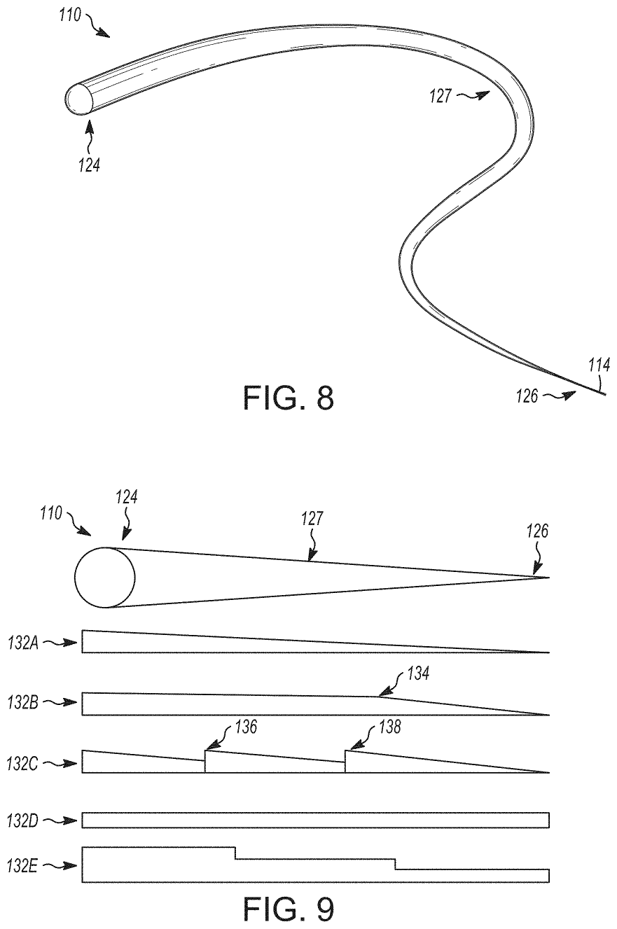

FIG. 8 is a perspective view of an exemplary compliant sensing tether 110, in accordance with embodiments of the present disclosure. In the illustrated embodiments, the proximal portion 124 may be less flexible than the distal portion 126. In at least some embodiments, the proximal portion 124 may be the portion of the compliant sensing tether 110 having the least flexibility and the distal portion 126 may be the portion of the compliant sensing tether 110 having the greatest flexibility. Additionally, or alternatively to having a distal portion 126 that is more flexible than the proximal portion 124, the compliant sensing tether 110 may have one or more of the following: a tensile strength less than 50 kPa, a toughness modulus less than 50 kPa, and/or a flexibility less than 50 kPa. Additionally, or alternatively, the distal portion 126 may have a compressive modulus between 1 kPa and 35 kPa.

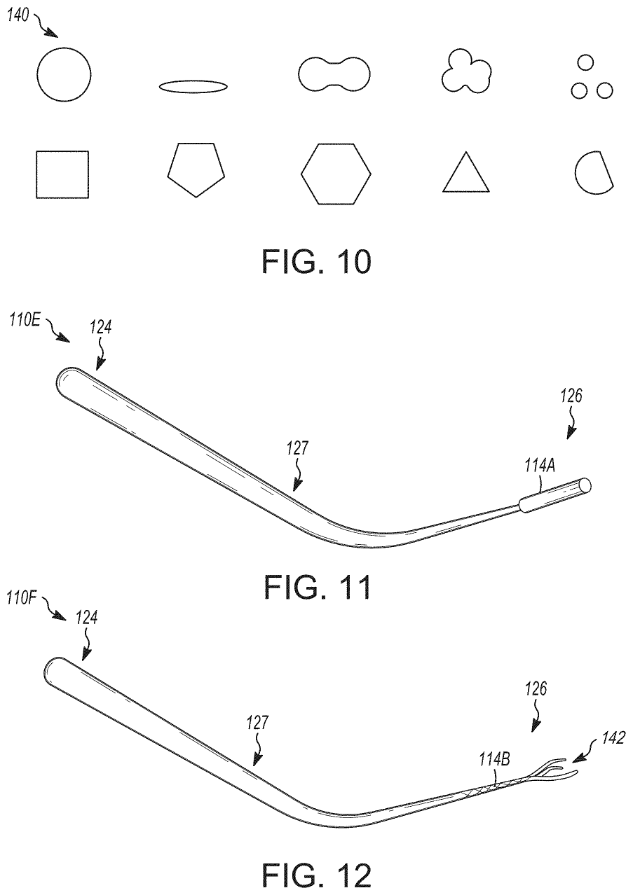

In the illustrated embodiment, the compliant sensing tether 110 has a circular cross section. In other embodiments, the compliant sensing tether 110 may have a cross sections having different shapes, such as the shapes depicted in FIG. 10. In the illustrated embodiment, the compliant sensing tether 110 has a stiffness that linearly decreases from the proximal portion 124, where the compliant sensing tether 110 couples to an electronic module 112, to the distal portion 126 including the sensing region 114. In at least some embodiments, the linearly decrease in stiffness (e.g., linear increase in flexibility) may be attributed to a decrease in diameter of the compliant sensing tether 110. As described in FIG. 9, however, the flexibility of the compliant sensing tether 110 may decrease in different manners than linearly.

FIG. 9 is a side view of an exemplary compliant sensing tether 110 and exemplary stiffness gradients 132 associated therewith, in accordance with embodiments of the present disclosure. The stiffness of the compliant sensing tether 110 may be varied according to any of the stiffness gradients 132 by varying the cross-sectional area of the compliant sensing tether 110 and/or varying the materials/construction (e.g., adding reinforcement) of the compliant sensing tether 110.

The first exemplary stiffness gradient 132A of the compliant sensing tether 110 decreases linearly (e.g., becomes linearly more flexible) from the proximal portion 124 of the compliant sensing tether 110 to the distal portion 126 of the compliant sensing tether 110. The second exemplary stiffness gradient 132B of the compliant sensing tether 110 decreases linearly at a first rate (e.g., becomes linearly more flexible at a first rate) from the proximal portion 124 to an intermediate point 134. At the intermediate point 134 to the distal portion 126, the stiffness of compliant sensing tether 110 decreases linearly at a second rate (e.g., becomes linearly more flexible at a second rate). The third exemplary stiffness gradient 132C decreases linearly (e.g., becomes linearly more flexible) from the proximal portion 124 to an intermediate point 134. At the first point 136, the stiffness of the compliant sensing tether 110 increases (e.g., becomes less flexible) to, for example, the stiffness at the proximal portion 124. Then, from the first point 136 to a second point 138, the stiffness of the compliant sensing tether 110 decreases linearly (e.g., becomes linearly more flexible). At the second point 138, the stiffness of the compliant sensing tether 110 increases (e.g., becomes less flexible) to, for example, the stiffness at the first point 136. Then, from the second point 138 to the distal portion 126, the stiffness of the compliant sensing tether 110 decreases linearly (e.g., becomes linearly more flexible). In the fourth exemplary stiffness gradient 132D stays constant from the proximal portion 124 to the distal portion 126. The fifth exemplary stiffness gradient 132E decreases in stepwise fashion (e.g., becomes linearly more flexible in a step-wise fashion) from the proximal portion 124 to the distal portion 126.

In at least some embodiments, the stiffness gradients illustrated in FIG. 9 may decrease in response to the compliant sensing tether 110 absorbing fluid. For example, each of the exemplary stiffness gradients may shift, linearly or nonlinearly, downward (i.e., the compliant sensing tether 110 may become more flexible), in response to fluid absorption by the compliant sensing tether 110. In embodiments, flexibility of the sensing tether 110 may increase as a result of fluid absorption (e.g., hydration), which allows for the sensing tether 110 and/or the sensing region 114 to progressively adjust to the properties of the surrounding tissue 115 until the sensing tether 110 and/or sensing region 114 properties match or substantially match (e.g., +/−5%, +/−10%, +/−15%) that of the surrounding tissue 115. Additionally, before fluid absorption, the sensing tether 110 may be stiffer, facilitating surgical implantation. Then, the sensing tether 110 and/or sensing region 114 may decrease its stiffness (e.g., increase its flexibility) in response to being hydrated to allow for better compliance matching to the surrounding tissue 115.

FIG. 10 are cross-sectional views of exemplary shapes 140 of a compliant sensing tether 110, in accordance with embodiments of the present disclosure. The compliant sensing tethers 110 discussed herein may have any one of the cross-sectional shapes depicted in FIG. 10. Examples of cross-sectional shapes include, but are not limited to: a circularly shape, an ovular shape, a random contiguous shape, a random dis-contiguous shape, various polygonal shapes (e.g., a triangle, a square, a pentagon, a hexagon), etc.

FIGS. 11-19 depict different exemplary compliant sensing tethers 110, features of which, either alone or in combination, may be incorporated into any of the compliant sensing tethers 110 disclosed herein. Similar to the embodiments depicted above, the proximal portions 124 of the compliant sensing tethers 110 may be communicatively coupled to an electronic module 112.

Referring to FIG. 11, a perspective view of an exemplary compliant sensing tether 110E is illustrated. The compliant sensing tether 110E includes electrode wires or optical fiber tips or a combination thereof arranged in, for example, a cylindrical shape to form the sensing region 114A. In at least some embodiments, the structure of the sensing region 114A may be used for the placement of additional system components (e.g., coating, membrane, film wrap, etc.). Additionally, or alternatively, the sensing region 114A may have high flexibility allowing deformation thereof. For example, one or several layers of protective material or substance, the attributes of which may be distinctively attuned to the target tissue and/or analyte(s) sensed by the sensing region 114A, may be added around the sensing structure region 114A.

FIG. 12 is a perspective view of even another exemplary compliant sensing tether 110F, in accordance with embodiments of the present disclosure. The illustrated embodiment includes a sensing region 114B having finger-like filaments 142. The compliant sensing tether 110F may include electrode wires or optical fibers or a combination thereof, arranged with a braided structure of conductive ePTFE, that extend from the proximal portion 124 to the distal portion 126 and terminate with a finger-like formation to form the sensing region 114B.

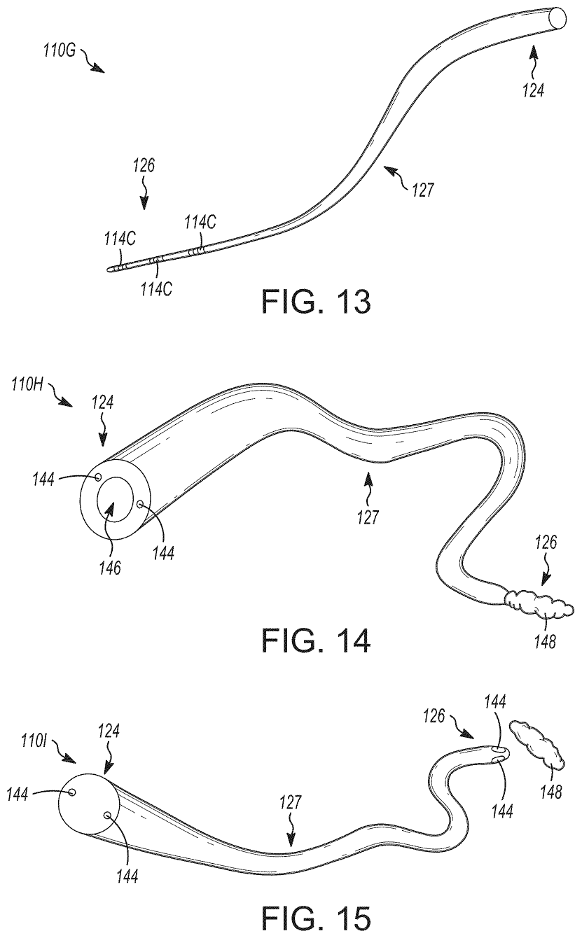

FIG. 13 is a perspective view of even another exemplary compliant sensing tether 110G, in accordance with embodiments of the present disclosure. The illustrated embodiment includes multiple sensing regions 114C distributed along a length of the distal portion 126 of the compliant sensing tether 110G. Each of the multiple sensing regions 114C may include electrodes, optical fibers, or combination thereof, and analyte sensing may be accomplished through electric and/or photonic sensing.

FIG. 14 is a perspective view of even another exemplary compliant sensing tether 110H, in accordance with embodiments of the present disclosure. The illustrated embodiment includes one or more optical fibers 144, embedded within the compliant sensing tether 110H, which extend from a proximal portion 124 to a distal portion 126. Additionally, or alternatively, the compliant sensing tether 110H may include a lumen 146 that extends from the proximal portion 124 to the distal portion 126. In at least some embodiment, hydrogel 148 can be delivered through the lumen 146 from the proximal portion 124 to and/or out of the distal portion 126. In embodiments, the hydrogel 148 may include one or more fluorescent probes that can be excited with a low energy photonic beam delivered by the adjacent optical fibers 144 to determine the presence and/or concentration of an analyte. The lumen 146 may also constitute a reservoir used to replenish the hydrogel 148 at the distal portion 126 of the compliant sensing tether 110.

FIG. 15 is a perspective view of even another exemplary compliant sensing tether 1101, in accordance with embodiments of the present disclosure. The illustrated embodiment includes one or more optical fibers 144, embedded within the compliant sensing tether 1101, which extend from a proximal portion 124 to a distal portion 126. The optical fibers 144 may be used to excite, with a low energy photonic beam delivered by the adjacent optical fibers 144, one or more fluorescent probes included in a hydrogel 148 that is detached from the distal portion 126 of the compliant sensing tether 110, in order to determine the presence and/or concentration of an analyte.

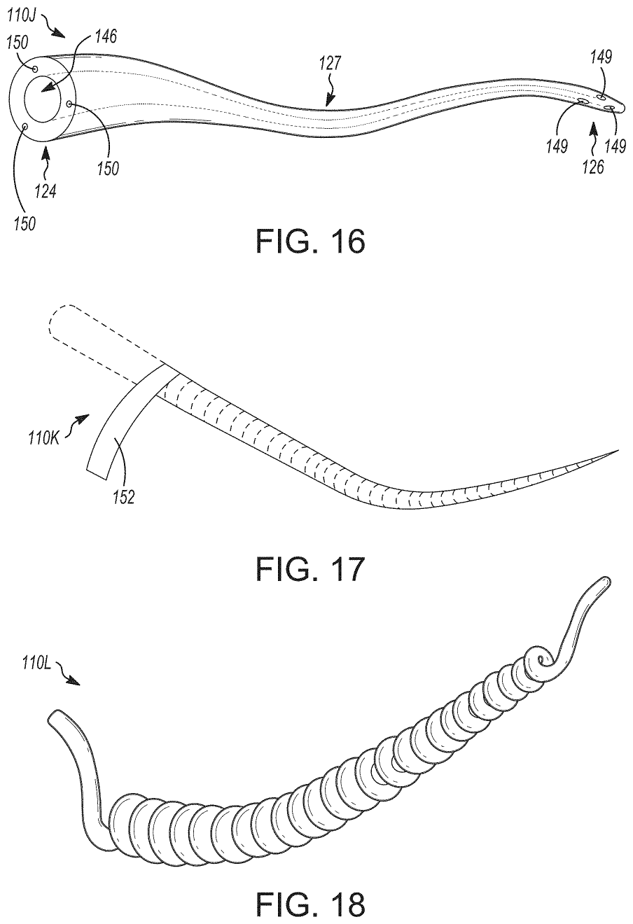

FIG. 16 is a perspective view of even another exemplary compliant sensing tether 110J, in accordance with embodiments of the present disclosure. The illustrated embodiment includes three electrodes (although more or fewer could be included) 149 arranged on the distal portion 126 of the compliant sensing tether 110J. The compliant sensing tether 110J may also include lumens 150 to connect the electrodes 149 to the electronic module 112. Additionally, or alternatively, the compliant sensing tether 110J may include lumen 146 for infusion of liquids or for electronic or photonic signal conduction from the proximal portion 124 to the distal portion 126.

FIG. 17 is a perspective view of even another exemplary compliant sensing tether 110K, in accordance with embodiments of the present disclosure. In the illustrated embodiment, a film 152 is wrapped around and/or may cover the compliant sensing tether 110. The film 152 may be used to reduce the foreign body response and/or to modify the flexibility of the compliant sensing tether 110K. In at least some embodiments, the film may be formed form a highly flexible and/or inert material, such as ePTFE, conductive ePTFE, polymers, silicone, hydrogel, as well electrodes and/or optical fibers.

FIG. 18 is a perspective view of even another exemplary compliant sensing tether 110L, in accordance with embodiments of the present disclosure. In the illustrated embodiment, the compliant sensing tether 110L is formed by a coiled structure. The coiled configuration of the compliant sensing tether 110L may enable compact packaging, facilitating surgical implantation of the compliant sensing tether 110L and subsequent deployment or relaxation of the compliant sensing tether 110L within host tissues 115.

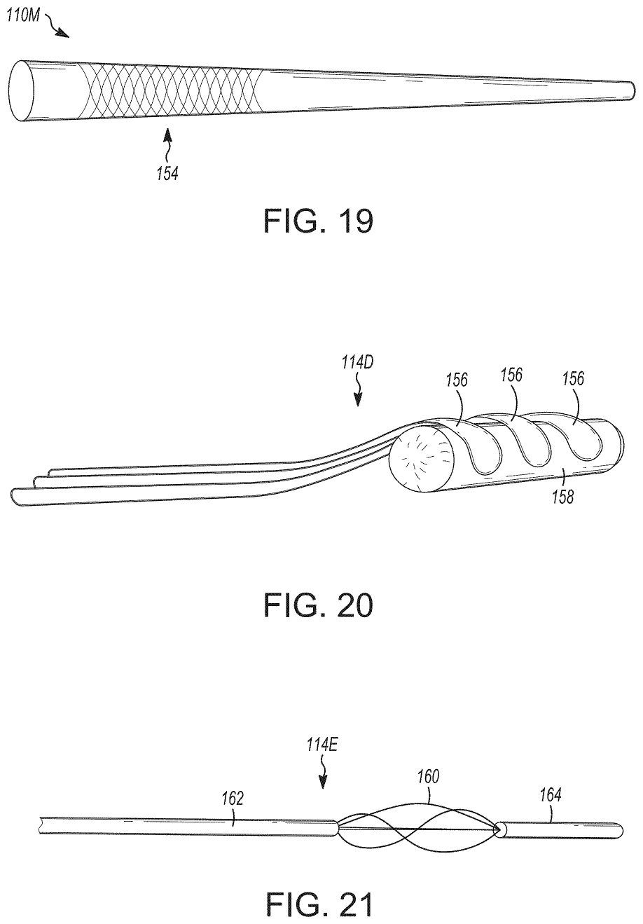

FIG. 19 is a perspective view of even another exemplary compliant sensing tether 110M, in accordance with embodiments of the present disclosure. The illustrated embodiment includes a reinforced section 154 including, for example, a reinforcement braid that may improve the mechanical performance of the compliant sensing tether 110M and/or facilitate varying the flexibility of the compliant sensing tether 110M.

FIG. 20 is a perspective view of an exemplary sensing region 114D of a compliant sensing tether 110, in accordance with embodiments of the present disclosure. In the illustrated embodiment, the sensing region 114D includes one or more ribbons 156 that may be wrapped onto a hydrogel form 158, thereby converting a flat construction into a cylindrical configuration. Each of the ribbons 156 may include electrode wires that are flattened or printed on a flexible polymer substrate and terminated around a polymeric support, such as a hydrogel, allowing the sensing region 114D to be a conductive sensing region.

FIG. 21 is a perspective view of another exemplary sensing region 114E of a compliant sensing tether 110, in accordance with embodiments of the present disclosure. In the illustrated embodiment, the sensing region 114E includes electrodes 160 arranged between a proximal portion 162 of a compliant sensing tether 110 and a distal portion 164 of a compliant sensing tether 110. This arrangement may help protect the electrodes 160 so the useful life of the compliant sensing tether 110 is prolonged.

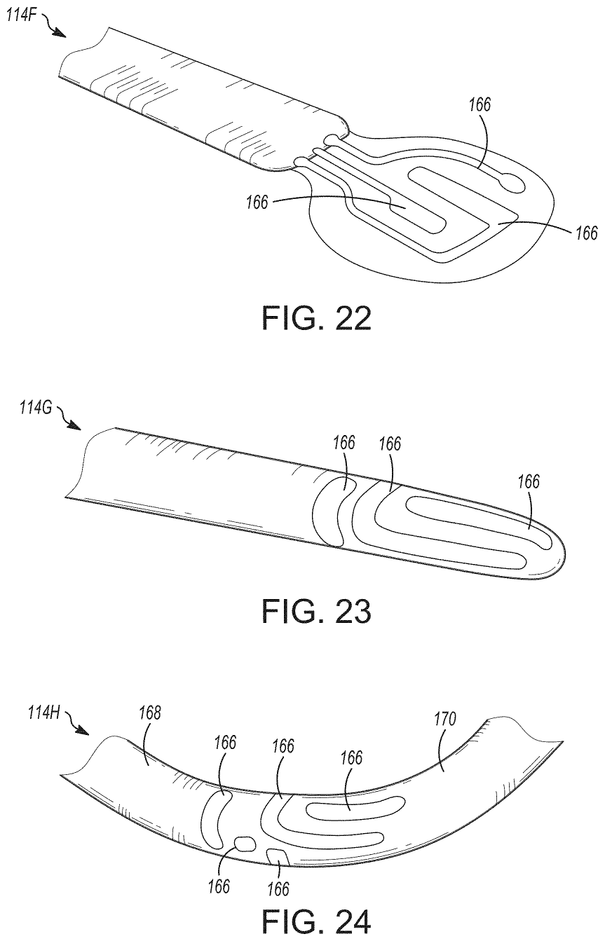

FIG. 22 is a perspective view of another exemplary sensing region 114F of a compliant sensing tether 110, in accordance with embodiments of the present disclosure. While three electrodes 166 are depicted, the sensing region 114F may include more or fewer electrodes 166. In the illustrated embodiment, the sensing region 114F is flat, arranged at a distal portion 126 of a compliant sensing tether 110, and includes a combination of electrodes 166 of various sizes, which may include a functional surface area and placement that is based on the analyte(s) being sensed.

FIG. 23 is a perspective view of another exemplary sensing region 114G of a compliant sensing tether 110, in accordance with embodiments of the present disclosure. The sensing region 114G may include a combination of electrodes 166 of various sizes. While three electrodes 166 are depicted, the sensing region 114G may include more or fewer electrodes 166. In the illustrated embodiment, the sensing region 114G is cylindrical, arranged at a distal portion 126 of a compliant sensing tether 110, and includes a combination of electrodes 166 of various sizes, which may include a functional surface area and placement that is based on the analyte(s) being sensed.

FIG. 24 is a perspective view of even another exemplary sensing region 114H of a compliant sensing tether 110, in accordance with embodiments of the present disclosure. The sensing region 114H may include a combination of electrodes 166 of various sizes. In embodiments, the sensing region 114H may include fewer or more electrodes 166 than the number of electrodes depicted. In the illustrated embodiment, the sensing region 114H is cylindrical, arranged between proximal 168 and distal 170 portions of a compliant sensing tether 110, and includes a combination of electrodes 166 of various sizes, which may include a functional surface area and placement that is based on the analyte(s) being sensed.

The embodiments disclosed herein have been described above both generically and regarding specific embodiments. It will be apparent to those skilled in the art that various modifications and variations can be made in the embodiments without departing from the scope of the disclosure. Thus, it is intended that the embodiments cover the modifications and variations of this disclosure provided they come within the scope of the appended claims and their equivalents.

1. An implantable biosensor configured to be implanted into tissue of a subject at an implantation site, the implantable biosensor comprising:
an electronic module;
a compliant sensing tether extending from the electronic module, the compliant sensing tether comprising a proximal portion coupled to the electronic module, a distal portion spaced apart from the electronics module, and an intermediate portion joining the proximal portion to the distal portion;
the proximal portion having a first flexibility and the distal portion having a second flexibility, the second flexibility of the distal portion being greater than the first flexibility of the proximal portion; and
the distal portion comprising a sensor configured to sense a signal corresponding to an analyte of the subject, wherein the signal corresponding to the analyte is transferred to the electronics module via the compliant sensing tether.

2. A method for monitoring an analyte of a subject, the method comprising:
inserting an implantable biosensor into an implantation site of the subject, the implantable biosensor comprising:
an electronic module;
a compliant sensing tether extending from the electronic module, the compliant sensing tether comprising a proximal portion coupled to the electronic module, a distal portion spaced apart from the electronics module, and an intermediate portion joining the proximal portion to the distal portion;
the proximal portion having a first flexibility and the distal portion having a second flexibility, the second flexibility of the distal portion being greater than the first flexibility of the proximal portion;
the distal portion comprising a sensor configured to sense a signal corresponding to an analyte of the subject;
sensing, by the sensor, the signal corresponding to the analyte of the subject; and
transferring, via the compliant sensing tether, the signal to the electronics module.

3. The method of claim 2, further comprising transmitting, by the electronic module, the signal to an external device.

4. The method of claim 2, further comprising analyzing, by the electronic module, the signal to determine an amount of analyte in the subject.

5. The biosensor of claim 1, the second flexibility of the distal portion being substantially equal to or less than a predetermined flexibility of the tissue at the implantation site.

6. The biosensor of claim 1, the compliant sensing tether having a stiffness gradient that decreases nonlinearly from the proximal portion to the distal portion.

7. The biosensor of claim 1, the compliant sensing tether having a stiffness gradient that decreases linearly from the proximal portion to the distal portion.

8. The biosensor of claim 1, the compliant sensing tether having a flexibility that increases in response to fluid absorption by the compliant sensing tether.

9. The biosensor of claim 1, the distal portion comprising a plurality of sensors.

10. The biosensor of claim 1, the distal portion being formed from ePTFE.

11. The biosensor of claim 1, the electronics module comprising an antenna, a battery, and a circuit board.

12. The biosensor of claim 1, the compliant sensing tether having a stepped compliance.

13. The biosensor of claim 1, the compliant sensing tether having one or more of the following characteristics: a tensile strength less than 50 kPa, a toughness modulus less than 50 kPa, and a flexibility less than 50 kPa.

14. The biosensor of claim 1, the distal portion having a compressive modulus less than 35 kPa.

15. The biosensor of claim 1, the compliant sensing tether configured to dispose a hydrogel proximate to the distal portion of the compliant sensing tether.

16. The biosensor of claim 15, the electronic module configured to sense a fluorescence of the hydrogel.

17. The biosensor of claim 1, the compliant sensing tether being separate from the electronic module and wherein the compliant sensing tether transmits sensor signals to the electronic module.

18. The biosensor of claim 1, the compliant sensing tether being separable from the electronic module.

19. The biosensor of claim 1, the compliant sensing tether being coated in a hydrogel.

20. The biosensor of claim 1, the implantable biosensor being incorporated into a therapeutic drug infusion pump.

21. A method of treatment using an implantable biosensor, the method comprising:
receiving sensed signals from the implantable biosensor implanted in a subject;
the implantable biosensor comprising:
an electronic module;
a compliant sensing tether extending from the electronic module, the compliant sensing tether comprising a proximal portion coupled to the electronic module, a distal portion spaced apart from the electronics module, and an intermediate portion joining the proximal portion to the distal portion;
the proximal portion having a first flexibility and the distal portion having a second flexibility, the second flexibility of the distal portion being greater than the first flexibility of the proximal portion; and
the distal portion comprising a sensor configured to sense a signal corresponding to an analyte of the subject, wherein the signal corresponding to the analyte is transferred to the electronics module via the compliant sensing tether;
processing the received signals to determine concentration of the analyte; and
sending a signal to a therapy device to provide treatment based on the determined concentration.

22. The method of claim 21, further comprising implanting the implantable biosensor in the subject.
